(19) United States
(12) Patent Application Publication
(10) Pub. No.: US 2017/0258331 A1
(43) Pub. Date: Sep. 14, 2017

(54) IMAGING DEVICE

(71) Applicant: SHIMADZU CORPORATION, KYOTO (JP)

(72) Inventor: Hiroyuki TSUMATORI, KYOTO (JP)

(73) Assignee: SHIMADZU CORPORATION, KYOTO (JP)

(21) Appl. No.: 15/509,530

(22) PCT Filed: Jul. 8, 2015
(86) PCT No.: PCT/JP2015/069598

§ 371 Date: Mar. 8, 2017

(30) Foreign Application Priority Data
Sep. 8, 2014 (JP) 2014-182536

(51) Int. Cl. A61B 5/00; G01N 21/359; G01N 21/64

(52) U.S. Cl. CPC A61B 5/0071; A61B 5/0077; G01N 21/6428; G01N 21/359; G01N 2021/6439

(57) ABSTRACT

An imaging device includes a visible light source 22, an excitation light source 23 that irradiates an excitation light to excite indocyanine green administered to a patient; an evaluation light source 24 that irradiates the light having the wavelength corresponding to the fluorescence from indocyanine green; a aperture mechanism 60 that narrows only visible light between visible light and fluorescence; a camera 21 capable of detecting fluorescence and visible light; a focal adjustment mechanism 70 that executes focal adjustment of the camera 21 using the light irradiated from the evaluation light source 24.

### CROSS REFERENCE TO RELATED APPLICATIONS

This application relates to and claims §371 priority from SN PCT/JP2015/069598 filed Jul. 8, 2015, the entire contents of which are incorporated herein by reference, which in turn claims priority to JP Ser. No. 2014-182536 filed Sep. 8, 2014.

### BACKGROUND OF THE INVENTION

Field of the Invention

The present invention relates to an imaging device that irradiates excitation light to the fluorescent dye (fluorophore) injected into the subject and images fluorescences emitted from the fluorophore therein.

### Background of the Invention

Recently, the method called near-infrared fluorescence imaging is applied in a surgery. According to the near-infrared fluorescence imaging, indocyanine green (ICG) as a fluorophore is injected into an affected area. And when near-infrared light as an excitation light having wavelength approximately 600-850 nm is irradiated to indocyanine green, indocyanine green emits fluorescence in the near-infrared region in which the peak of the wavelength is approximately 750-900 nm. Such fluorescence is imaged by a camera that can detect the near-infrared light, and then the image thereof is displayed on a display such as a liquid crystal (LC) display panel and so forth for viewing. According to such near-infrared fluorescence imaging, blood vessels and lymphatic vessels and so forth, which are around 20 mm below the body surface, can be observed.

Patent Document 1 discloses a data collection method for collecting the data of the area undetectable in the affected cancer area distribution image as the sub-affected cancer area data despite detectable in the strength-distribution image using the near-infrared fluorescence by comparing the near-infrared fluorescence strength-distribution image obtained by irradiating the excitation light of indocyanine green to the body's target organ in which indocyanine green is injected, and the cancer affected area distribution image obtained by applying an X-ray, a nuclear magnetic resonance or an ultrasound wave relative to the target organ before injecting indocyanine green.

In addition, Patent Document 2 discloses a surgery assist method using irradiating an excitation light and visible light alternately to the subject to which an angiographic contrast agent is administered, acquiring obtaining a fluorescent image and a visible image alternately to which the excitation lights are irradiated by an imaging means, extracting the blood vessel image by conducting a threshold processing relative to the fluorescent image using a predetermined threshold value and generating a fused image by superimposing the visible image and the extracted blood vessels image.

### RELATED PRIOR ART DOCUMENTS

### Patent Document

Patent Document 1 PCT International Publication No. WO2009-139466

Patent Document 2: JP Patent Published 2009-226072

Patent Document 3: PCT International Publication No. WO2011-007461

### ASPECTS AND SUMMARY OF THE INVENTION

### Problems to be Solved by the Invention

According to Patent Document 2, when the fused image is generated by combining the visible image and the fluorescent image, after coloring the black and white fluorescent image, the visible image and the colored fluorescent image are combined by e.g., using the method for an additive fusion and the screen fusion and so forth. A camera that images such visible image and the fluorescent image comprises; a sensor for fluorescence, such as CCD and CMOS that detects fluorescence, a sensor for visible light, such as CCD and CMOS that detects visible light, and an optical isolation member, such as a wavelength selection filter that allows the coaxially incident fluorescence and visible light, which are reflected from the subject, into the camera, selectively into the sensor for fluorescence and the sensor for visible light, i.e., multi-board type, can be applied.

In addition, if the strength of the visible light is too strong, halation and so forth takes place, so that the aperture mechanism for the visible light is installed in the front side (subject side) of the optical isolation member. In addition, a focal adjustment mechanism for the focal points of the incident visible light and fluorescence so as to coincide with the sensor for visible light and the sensor for fluorescence is installed in the front side of the optical isolation member. At this time, fluorescence would not emit unless the excitation light is irradiated to the subject after the administration of indocyanine green under the examination state, so that above focal adjustment is executed generally by applying the visible light.

FIG. 7 is a schematic view illustrating narrowing the visible light by an aperture mechanism. Further, referring to FIG. 7, the visible light is indicated by the solid line and the fluorescence is indicated by the broken line.

The visible light and the fluorescence are coaxially incident into the camera. At this time, the visible light is narrowed by the aperture mechanism 160 that narrows only visible light. And the visible light and the fluorescence are focused on the focal position F by the movable lens 171 constituting the focal adjustment mechanism. The visible light and the fluorescence are detected by the sensor for visible light and the sensor for fluorescence, not shown in FIG., at the focal position F.

At this time, referring to FIG. 7, when the visible light is not narrowed by the aperture mechanism 160, the focal depth of the visible light and the fluorescence is in the relatively small area as indicated by the sign b. Specifically, under such circumstance, the focal depth is shallow. In contrast, referring to FIG. 7, when the visible light is narrowed by the aperture mechanism 160 in order to adjust an amount of the visible light, the focal depth of the visible light is relatively large area as indicated by the sign a. Specifically, the focal depth of the visible light becomes deep.

Under such circumstance, when the focal adjustment is executed by using the visible light, the focal point of the visible light can be adjusted in the focal depth of the visible light, i.e., within the area indicated by the sign a in FIG. 7. On the other hand, even when the visible light is narrowed, the focal depth of the fluorescence would not vary, so that the fluorescence can be focused only within the area indicated by the sign b in FIG. 7. Accordingly, it is problematic that when the visible light is applied to adjust the focal point, and even when the focal point is within the area indicated by the sign a in FIG. 7, but outside the area indicated sign b in FIG. 7; the fluorescence cannot be focused on the sensor for the fluorescence.

The present invention is to provide solutions for the above set forth problems and provides an imaging device capable of accurately executing focal point adjustment of visible light and fluorescence when the visible light and the fluorescence are focused simultaneously and even when the visible light is narrowed.

### Means for Solving the Problem

According to the first invention, an imaging device comprises: an excitation light source that irradiates the excitation light, which excites a fluorophore administered into the subject, to a subject; a visible light source that irradiates the visible light to the subject; an aperture mechanism that narrows the visible light irradiated from the visible light source and reflected from the subject's surface; a camera capable of detecting the fluorescence and the visible light, wherein the fluorescence that is emitted from the fluorophore due to the irradiation of the excitation light, and the visible light that is reflected from the subject's surface are imaged; and further comprises: a third light source that irradiates the light having wavelength covering (comparable) at least the fluorescence; and a focal adjustment mechanism that executes the focal adjustment for the camera by utilizing the light irradiated from the third light source.

According to aspect of the second invention, the third light source is an evaluation light source that irradiates the light having the wavelength covering at least the fluorescence, wherein whether said camera is working normally and appropriately or not is determined by imaging the image of the irradiation area using said camera.

According to aspect of the third invention, wherein the aperture mechanism is the aperture mechanism that narrows only the visible light between the fluorescence and the visible light.

According to aspect of the fourth invention, the camera comprises; a sensor for fluorescence that detects fluorescence, a sensor for visible light that detects visible light, and an optical isolation member that allows the coaxially incident fluorescence and visible light into the camera and selectively into the sensor for fluorescence and the sensor for visible light.

According to aspect of the fifth invention, the excitation light and the fluorescence are the near-infrared light.

### Effect of the Invention

According to the inventions from the first invention to the fifth invention, when each focal point of the visible light and the fluorescence is adjusted simultaneously and even when the visible light is narrowed, the focal adjustment of the visible light and the fluorescence can be accurately executed by utilizing the light irradiated from the third light source for the focal adjustment.

The above and other aspects, features and advantages of the present invention will become apparent from the following description read in conjunction with the accompanying drawings, in which like reference numerals designate the same elements.

### BRIEF DESCRIPTION OF THE DRAWINGS

FIG. 1 is a schematic view of an imaging device according to the present invention.

FIG. 2 is a perspective view of a lighting-imaging element 12.

FIG. 3 is a schematic front view of the lighting-imaging element 12.

FIG. 4 is a schematic front view illustrating inside of the lighting-imaging element 12 excluding the light source.

FIG. 5A, 5B are schematic views illustrating an aperture mechanism 60.

FIG. 6 is a block diagram illustrating the main control system of the imaging device of the present invention.

FIG. 7 is a schematic explanation view illustrating narrowing the visible light by an aperture mechanism.

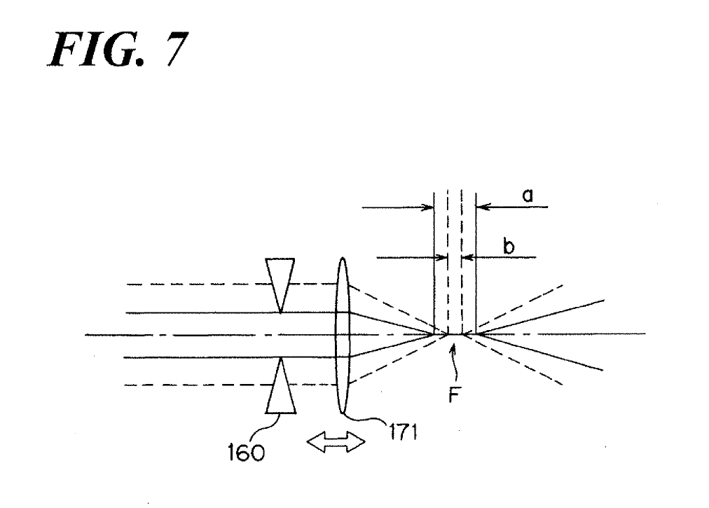

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

It will be further understood by those of skill in the art that the imaging apparatus and devices and the elements herein, without limitation, and including the sub-components such as operational structures, circuits, PC boards, communication pathways, and related elements, control elements of all kinds in a physical sense however described, display circuits and display systems and elements, any necessary driving elements, inputs, sensors, detectors, electronic memory elements, processors and any combinations of these structures etc. as will be understood by those of skill in the art as also being identified as or capable of operating the systems and devices and subcomponents noted herein and structures that accomplish the functions noted herein without restrictive language or label requirements since those of skill in the art are well versed in related imaging device technology, computer and operational controls and technologies of radiographic devices and all their sub components, including various circuits and combinations of circuits without departing from the scope and spirit of the present invention.

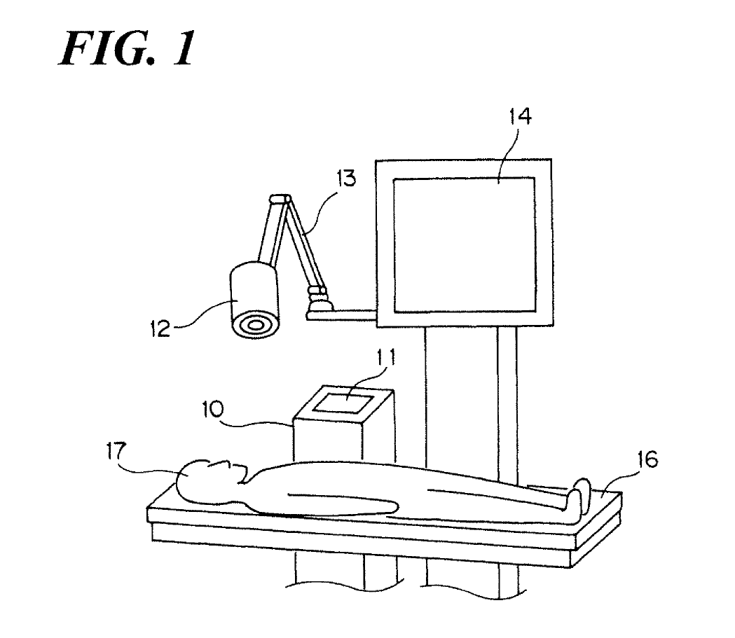

The inventor sets forth Embodiments of the present invention based on the following FIGs. FIG. 1 is a schematic view of an imaging device according to the present invention.

The imaging device of the present invention further comprises: an input element 11 such as a touch panel and so forth; a main body 10 including a control element 30 inside, set forth later; a lighting-imaging element 12 movably supported by an arm 13; a display element 14 including such as a liquid crystal display panel; and a treatment table 16 on which a subject (patient) 17 would be loaded. In addition, the lighting-imaging element 12 is not limited to be supported by the arm 13, but may be carried in an operator's hands.

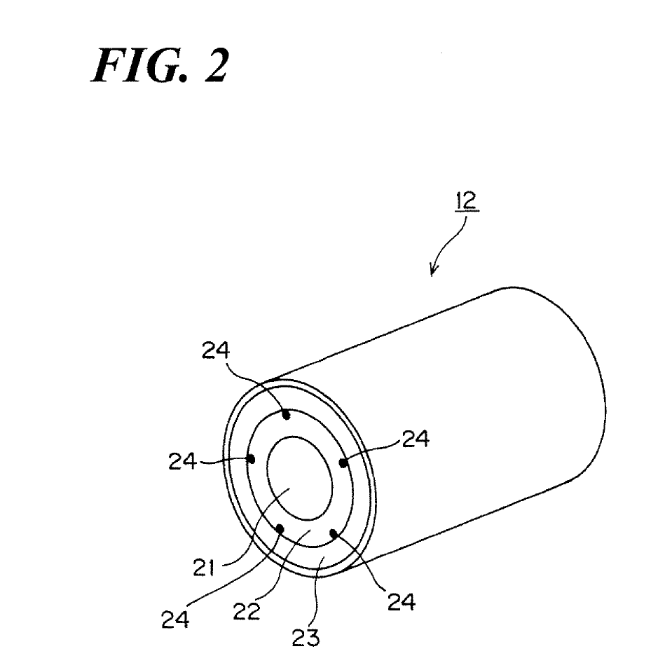

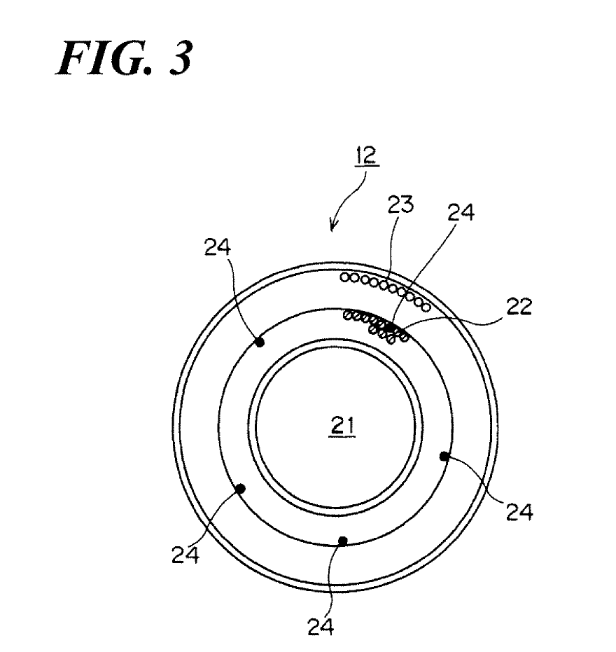

FIG. 2 is a perspective view of a lighting-imaging element 12. FIG. 3 is a schematic front view of the lighting-imaging element 12.

The lighting-imaging element 12 comprises: a camera 21 that can detect near-infrared light and visible light; a visible light source 22 consisting of one or plural LEDs that are mounted to the circumference of the camera 21; an excitation light source 23 consisting of one or plural LEDs that are mounted to the circumference of the visible light source 22; and a confirmation light source 24 (corresponding to the third light source) consisting of one or plural LEDs (5 in the present Embodiment) that are mounted in many LEDs constituting the visible light source. The visible light source 22 irradiates visible light. The excitation light source 23 irradiates near-infrared light having the wavelength 760 nm, which is an excitation light to excite indocyanine green. In addition, the evaluation (confirmation) light source 24 irradiates near-infrared light having the wavelength 810 nm, which is close to the wavelength of the fluorescence emitted from indocyanine green. Further, referring to FIG. 3, a part of many LEDs foaming the visible light source 22 and a part of many LEDs forming the excitation light source 23 are not shown in the FIG.

In addition, near-infrared light having the wavelength approximately 800 nm as the peak emitted from indocyanine green is invisible for a human naked eye. Under such circumstance, conventionally a card-like or a rod-like jig mixed with indocyanine green is prepared, and the excitation light is irradiated to such jig, and then the camera 21 images the jig, to which the excitation light is irradiated, so that the operation of the camera 21 can be confirmed. Therefore, not only an exclusive jig is required, but also complex operations, in which the jig must be prepared and the imaging operation is executed, are required.

Accordingly, relative to the camera 21, the imaging device of the present invention comprises an evaluation light source 24 that irradiates the near-infrared light having the wavelength 810 nm close to the near-infrared light having the peak of the wavelength at approximately 810 nm emitted from indocyanine green is additionally installed and the operation confirmation of the camera 21 is conducted by lighting the evaluation light source 24. And the near-infrared light irradiated from the evaluation light source 24 is utilized to execute the focal adjustment of the camera 21.

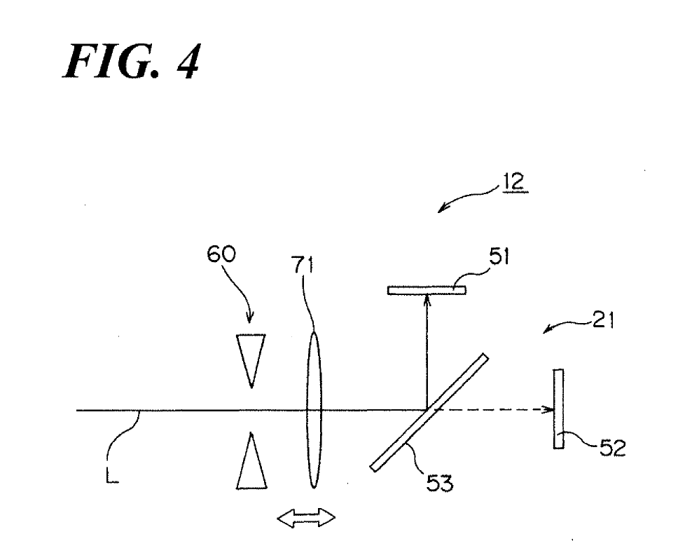

FIG. 4 is a schematic front view illustrating inside of the lighting-imaging element 12 excluding the light source.

Relative to the lighting-imaging element 12, the visible light between the visible light and the fluorescence incident coaxially along the light axis L thereof is narrowed by the aperture mechanism 60. On the other hand, the fluorescence passes through the aperture mechanism 60 without being narrowed. And the visible light and the fluorescence reach to a wavelength selection filter 53 after passing through the movable lens 71 forming the focal adjustment mechanism 70 set forth later. The visible light of the visible light and the fluorescence incident coaxially is reflected from the wavelength selection filter 53 and is incident on the sensor 51 for the visible light of the CCD or CMOS and so forth forming the camera 21. The fluorescence of the visible light and the fluorescence in coaxial passes through the wavelength selection filter 53 and is incident on the sensor 52 for the fluorescence of the CCD or CMOS and so forth forming the camera 21. At this time, the visible light is focused on the sensor 51 for visible light and the fluorescence is focused on the sensor 52 for fluorescence due to the action of the focal adjustment mechanism 70 including the movable lens 71.

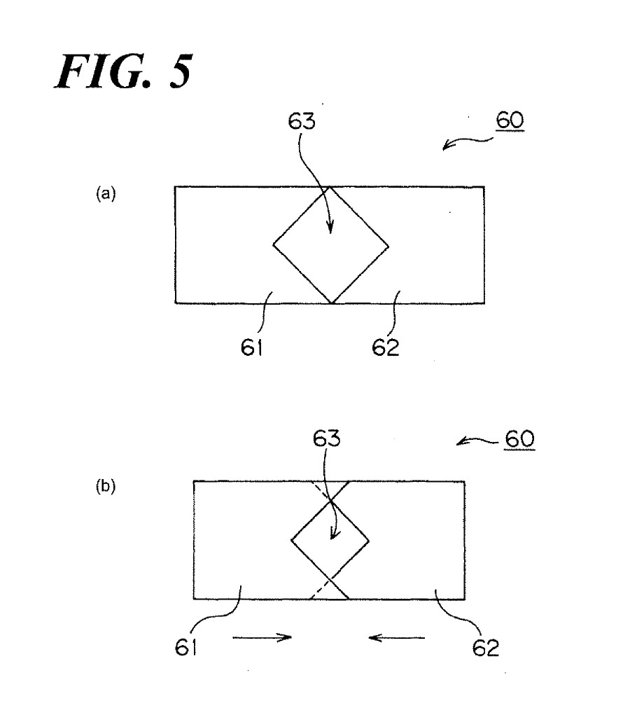

FIG. 5 is a schematic view illustrating the aperture mechanism 60 set forth above.

The aperture mechanism 60 set forth above comprises a pair of filter members 61, 62. Such filter members 61, 62 block visible light but pass fluorescence. V-like cut is formed at the end of each filter member 61, 62 and the filter members 61, 62 are arranged so that such V-like cuts can face each other. In addition, such filter members 61, 62 are movable in both coming close direction and getting away directions each other. Accordingly, referring to FIG. 5A, when the filter members 61, 62 are arranged isolated each other, the aperture area 63 is large and when the filter members 61, 62 are coming close from the location indicated in FIG. 5A, the aperture area 63 becomes smaller. Specifically, in accordance with the arrangement of the filter members 61, 62, the area size of the aperture area 63, through which the visible light passes, is variable, so that the visible light can be narrowed. On the other hand, fluorescence passes through the filter members 61, 62, so that the fluorescence is never narrowed by the aperture mechanism 60 regardless the area size of the aperture area 63.

Such aperture mechanism 60 is disclosed in PCT Published 2011-007461.

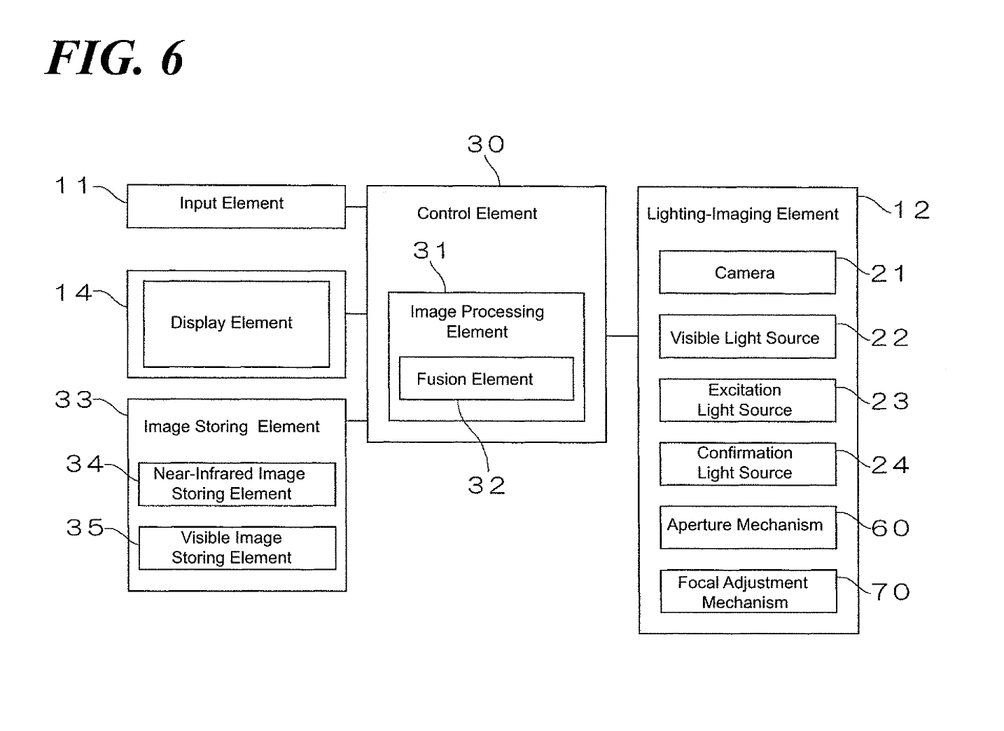

FIG. 6 is a block diagram illustrating the main control system of the imaging device according to the aspect of the present invention.

Further, the imaging device comprises: a CPU that executes the logic operation; a ROM that stores operation programs required to control the apparatus; a RAM that stores temporally the data and so forth when controlling; and so forth; and further comprises: a control element 30 that controls the entire apparatus. The control element 30 comprises an image processing element 31 that executes various kinds of imaging processing set forth later. Further, the control element 30 is also connected to the input element 11 and the display element 14 set forth above. In addition, the control element 30 is connected to the lighting-imaging element 12 comprising the camera 21, the visible light source 22, the excitation light source 23, the evaluation light source 24, the aperture mechanism 60 and the focal adjustment mechanism 70. Further, the control element 30 is also connected to an image storing element 33 that stores images imaged by the camera 21. The image storing element 33 comprises a near-infrared light image storing element 34 that stores the near-infrared image and a visible image storing element 35 that stores the visible images. Meantime, instead of comprising the near-infrared light image storing element 34 and the visible image storing element 35, a fused image storing element that stores the fused images combining the visible image and the near-infrared image can be equipped.

Hereafter, the inventors set forth an operation when a surgical operation is performed by using the imaging device according to the aspect of the present invention. In addition, as set forth below, the case is set forth when the surgery is performed on the breast cancer of a patient 17.

When the surgery of the breast cancer is performed using the imaging device according to the aspect of the present invention, firstly the evaluation light source 24 is turned on and an image is imaged by the sensor 52 for fluorescence of the camera 21 at this time. The evaluation light source 24 irradiates near-infrared light having the wavelength 810 nm, which is close to the wavelength of the fluorescence emitted from indocyanine green. Such near-infrared light is invisible for the human naked eye to confirm. On the other hand, the evaluation light source 24 irradiates the near-infrared light having the wavelength 810 nm and an image of the irradiation area is imaged by the camera 21, and further when the camera 21 is working normally, the image of the area to which the near-infrared light is irradiated is imaged by the camera 21 and such image is displayed on the display element 14. Accordingly, the operation of the camera 21 can be easily validated.

In addition, under such condition, the visible light that is irradiated from the visible light source 22 and is reflected from the patient 17 is narrowed by using the aperture mechanism 60, so that the incident light amount of visible lights on the sensor 51 for the visible light forming the camera 21 can be adjusted.

Further, under such condition, the focal adjustment of the camera 21 is executed. At this time, the sensor 52 for fluorescence of the camera 21 images the near-infrared light that is irradiated from the evaluation light source 24 and reflected from the patient 17 and the focal adjustment mechanism 70 executes a focal adjustment based on the obtained image by moving the movable lens 71. At this time, the near-infrared light that is irradiated from the evaluation light source 24 and reflected from the patient 17 is not narrowed by the aperture mechanism 60, so that the focal depth does not vary. Therefore, the focal adjustment of the visible light and the near-infrared light (i.e., fluorescence) can be executed accurately.

When the above preparation steps are complete, indocyanine green is injected to the breast of the patient 17 lying on the treatment table 16. And the near-infrared light is irradiated to the subject including the affected area from the excitation light source 23, and also the visible light is irradiated to the same from the visible light source 22. In addition, the near-infrared light irradiated from the excitation light source 23 is the near-infrared light having the wavelength 760 nm workable as the excitation light for indocyanine green to emit fluorescence. Accordingly, indocyanine green emits the fluorescence in the near-infrared light area of which the peak of the wavelength is at approximately 800 nm.

Then the affected area of the patient 17 and the surrounding thereof are imaged by the camera 21. The camera 21 can detect both near-infrared light and visible light. Referring to FIG. 6, the near-infrared image and the visible image imaged by the camera 21 are forwarded to the imaging processing element 31. The image processing element 31 converts the near-infrared image and the visible image to the displayable image data on the display 14. The near-infrared image data are stored in the near-infrared image storing element 34 of the image storing element 33. The visible image data are stored in the visible image storing element 35 of the image storing element 33.

In addition, an image fusion element 32 of the image processing element 31 generates a fused image combining the visible image and the near-infrared image. And the image processing element 31 displays either simultaneously or selectively the near-infrared image, the visible image and the fused image on the display 14.

In addition, according to the aspect of the above Embodiments, the near-infrared light having the wavelength 760 nm is applied as the excitation light source 23, but also near-infrared light having any wavelength between approximately 700 nm and approximately 900 nm as the excitation light source 23 can be applied as far as indocyanine green could be excited.

In addition, according to the aspect of the above Embodiments, the near-infrared light having the wavelength 810 nm is applied as evaluation light source 24, but also any evaluation light source 24 as far as near-infrared light having any wavelength between approximately 750 nm and approximately 900 nm, which is an emission wavelength of indocyanine green, can be applied.

In addition, according to the aspect of the above Embodiments, the inventors set forth the case in which indocyanine green is applied as a material containing a fluorophore and the fluorescence of the near-infrared area having the peak of the wavelength at approximately 800 nm is emitted from indocyanine green by irradiating the near-infrared light having the wavelength 760 nm to such indocyanine green as an excitation light, but a light other than near-infrared light can be applied.

For example, 5-ALA (standing for 5-Aminolevulinic Acid) can be applied as a fluorophore. When 5-ALA is applied, the 5-ALA administered inside the patient's body 17 changes to protoporphyrin IX/PpIX which is a fluorescent substance. When visible light having the wavelength approximately 400 nm is irradiated toward protoporphyrin, red visible light as a fluorescence is emitted from protoporphyrin. Accordingly, when 5-ALA is applied, an excitation light source that irradiates visible light having the wavelength approximately 400 nm may be used, and also a evaluation light source that can irradiate red visible light as the fluorescence emitted from protoporphyrin can be applied.

### REFERENCE OF SIGN

10 Main body
11 Input element
12 Lighting-imaging element
13 Arm
14 Display element
16 Treatment table
17 Subject
21 Camera
22 Visible light source
23 Excitation light source
24 Evaluation (Confirmation) light source
30 Control element
31 Image processing element
33 Image storing element
34 Near-Infrared image storing element
35 Visible light image storing element
51 Sensor of visible light
52 Sensor of fluorescence
53 Wavelength selection filter
60 Aperture mechanism
61 Filter
62 Filter
63 Aperture area
70 Focal adjustment mechanism
71 Movable lens

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

1. An imaging device, comprising:
an excitation light source that irradiates an excitation light to a subject;
wherein the excitation light excites a fluorophore administered into said subject to emit a fluorescence;
a visible light source that irradiates a visible light to said subject;
an aperture mechanism that narrows the visible light irradiated from said visible light source and that is reflected from a surface of said subject; and
a camera capable of detecting said fluorescence and the visible light;
wherein said fluorescence is emitted from said fluorophore due to the irradiation of the excitation light and said visible light is reflected from said subject's body;
and further comprising:
a third light source that irradiates a light having a wavelength covering at least said fluorescence; and
a focal adjustment mechanism that executes a focal adjustment for a camera by utilizing said light irradiated from said third light source.

2. The imaging device according to claim 1, wherein:
said third light source is a evaluation light source that irradiates the light having the wavelength covering at least said fluorescence, and capable of evaluating
whether said camera is working normally and appropriately or not is determined by an imaging of the image of an irradiation area using said camera.

3. The imaging device according to claim 2, wherein:
said aperture mechanism is the aperture mechanism that narrows only said visible light between said fluorescence and said visible light.

4. The imaging device, according to claim 2, wherein said camera further comprises:
a sensor for fluorescence that detects fluorescence, a sensor for visible light that detects visible light, and
an optical isolation member that allows the coaxially incident fluorescence and visible light into said camera and selectively into said sensor for fluorescence and said sensor for visible light.

5. The imaging device, according to claim 1, wherein:
said excitation light and said fluorescence are a near-infrared light.
